(19) United States
(12) Patent Application Publication
(10) Pub. No.: US 2019/0127777 A1
(43) Pub. Date: May 2, 2019

(54) Attribute Sieving and Profiling By Pooled Sanger Sequencing

(71) Applicant: BioInventors & Entrepreneurs Network, LLC, Warren, NJ (US)

(72) Inventors: Michael Seul, Basking Ridge, NJ (US); Ghazala Hashmi, Holmdel, NJ (US)

(21) Appl. No.: 15/822,186

(22) Filed: Nov. 26, 2017

Related U.S. Application Data
(60) Provisional application No. 62/580,784, filed on Nov. 2, 2017.

(51) Int. Cl. C12Q 1/6806; C40B 20/04; G01N 33/58; C12N 15/10; C40B 50/10; C40B 50/16

(52) U.S. Cl. CPC C12Q 1/6806; C40B 20/04; G01N 33/58; C12N 15/1065; C12Q 2563/179; C40B 50/16; C12Q 2537/143; C12Q 2537/159; C12Q 2537/165; C40B 50/10

(57) ABSTRACT

Disclosed is a novel method of allele profiling, or nucleic acid sieving, with pooled Sanger sequencing as a first (aka “screening”) stage; where the first step is: amplifying a single sequence, delineated by forward and reverse primers which may represent a single exon, or a segment thereof, or a contiguous stretch of multiple exons and introns. The amplicons produced from a pool of samples include the amplified sequence, and these are next converted into fragments in the standard Sanger labeling reaction. Ambiguities will appear as superposed peaks at any heterozygous position of interest, as the origin of the variant signal cannot be uniquely attributable to a specific sample, or samples, in the pool. These ambiguities may be resolved by the allele profiling process; or, resolution can be done with source-tagged primers generating source-tagged amplicons, which generate position shifts in labels, which can be decoded to resolve the ambiguities.

### BACKGROUND

Allele profiling (see US Publ'n No. 2015/0376693; U.S. Pat. No. 9,133,567, U.S. Ser. No. 15/202,035, all incorporated by reference) employs two reactions, the first of these a PCR (or other) amplification (“A”) reaction, and the second a discrimination (“D”) reaction. By incorporating source tags in first reaction products, allele profiling permits the pooling of these products as inputs to the second reaction to form a multiplicity of allele-specific second products that also incorporate marker tags identifying alleles. Allele profiling permits the simultaneous analysis of multiple alleles, over multiple exons, for multiple samples.

Sieving of nucleic acids (see US Publ'n No. 2015/0315568; U.S. Pat. No. 8,932,989, both incorporated by reference), rather than delivering results for all samples in a given sample set, permits the selection, from that set, of nucleic acids having (or not having) desired alleles or allele patterns. As with allele profiling, sieving relies on the incorporation of source and marker tags, into respective first and second reaction products.

Allele profiling and sieving, in addition to providing for the pooling of multiple first products into pooled second reactions, also provide for combining multiple samples in pooled first reactions. As the resulting pooled pools may leave unresolved ambiguities, these methods also provide a disambiguation step to identify allele configurations of samples in pools, or pools of pools, with unresolved allele configurations of interest (see US Publ'n Nos. 2015/0376693; 2015/0315568; U.S. Pat. Nos. 9,133,567; 8,932,989).

Sanger sequencing, in its prevalent commercial embodiment, comprises a PCR amplification reaction, to produce a single reaction product for each of usually two amplified alleles, a cleanup step to remove unused primers and dNTPs, followed by a “labeling” reaction to produce second products, in the form of fragments of all lengths, each terminated by (random) incorporation of base-specifically labeled, di-deoxy nucleotides. These second reaction products are then analyzed by capillary electrophoresis such that heterozygous samples produce two distinct peaks at any variable sequence position in which alleles differ in composition.

### SUMMARY

The present invention discloses an embodiment of allele profiling, or nucleic acid sieving, with pooled Sanger sequencing as a first (aka “screening”) stage. As with Sanger sequencing generally, this embodiment comprises amplifying a single sequence, delineated by forward and reverse primers which may represent a single exon, or a segment thereof, or a contiguous stretch of multiple exons and introns. The amplification products (“amplicons”) produced from a pool of samples each comprise the amplified sequence, and these are next converted into fragments in the standard Sanger labeling reaction. Ambiguities will arise in the subsequent analysis in the form of superposed peaks at any heterozygous position of interest, as the origin of the variant signal is not uniquely attributable to a specific sample, or samples, in the pool. These ambiguities may be resolved by the allele profiling process described in US Publ'n No. 2015/0376693; U.S. Pat. No. 9,133,567; and U.S. Ser. No. 15/202,035, with a smaller number, d, of samples per pool of first products, wherein d is an integer and d≥1, and wherein the pool of origin is identified by incorporating source tags into the first reaction products, to permit pooling of these first reaction products (as described for allele profiling in the foregoing references).

This present embodiment of allele profiling (and sieving) using pooled Sanger sequencing has the advantage that it can detect unanticipated variable sites in the amplified sequence. It retains the advantage of efficient disambiguation by the use of source tags enabling the pooling of first reaction products as inputs for the second (“discrimination”) reaction.

In a further embodiment, the invention provides for pooled Sanger sequencing of amplicons incorporating source tags to generate shifted sequence traces from pooled samples that permit the identification of alleles for at least two combined Sanger labeling reaction products. Source tags may differ in length or in base composition so as to induce known shifts in the predicted peak positions of interest, thereby permitting disambiguation, as shown herein.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIG. 1—a process flow diagram indicating a screening stage, with pooled Sanger sequencing, for detecting ambiguities in a delineated sub-sequence of first and second DNA molecules, and a disambiguation stage for identifying alleles of individual samples at positions giving rise to ambiguities.

FIG. 2—diagram showing allele configurations for first and second DNA samples, the alleles within a subsequence delineated by a pair of primers used for amplification.

FIG. 3—expected Sanger sequencing “reads” for pool of two samples comprising β-thalassemia mutations IVSI-5 (G>C) (top) and codon 8/9 (+G) (bottom).

FIG. 4—Sanger sequencing traces for a normal sample (top panel), for a sample homozygous for codon 8/9 (+G) (second-from-top panel), and for a pool comprising both samples (lowermost panel); the vertical highlight, in the second-from-top and in the lowermost panels, indicates the position of the insert.

FIG. 5—allele configurations for first and second DNA samples illustrating disambiguation for RHCE Exon 5: two different pairs of allele-specific forward primers, directed to two variable sites, and corresponding tagged reverse primers are indicated.

FIG. 6—depiction of the use of source tags for introducing integer peak shifts in the Sanger labeling reaction, for RHCE Exon 5: the insert (bottom right) illustrates the design of source-tags sharing a 5′ subsequence, here comprising the entire S8 tag sequence; the labeling primer is complementary to this shared subsequence.

FIG. 7—expected Sanger sequencing “reads” for pool of two source-tagged samples comprising β-thalassemia mutation codon 8/9 (+G); source tags S8 and S10 differ in length by 2 nucleotides

FIG. 8—expected Sanger sequencing “reads” for a pool of two source-tagged samples comprising β-thalassemia mutation IVSI-5 (G>C); source tags S8 and S10 differ in length by 2 nucleotides.

FIG. 9—expected Sanger sequencing “reads” for pool of two source-tagged samples comprising cystic fibrosis mutation G542X (c.1624 G>T); source tags S18 and S22 differ in length by 4 nucleotides

FIG. 10—Sanger sequencing traces for a normal sample (top panel), tagged with source tag S18, and for a sample homozygous for cystic fibrosis mutation G542X (c.1624 G>T), tagged with source-tag S22, and for a pool comprising both source-tagged samples; the vertical highlights indicate the variable positions in the S18 and S22-tagged samples (top and second-from-top panel, respectively), and in the compound trace (bottom panel); the insert gives a magnified version of the boxed segment in the compound trace. NOTE that, because the origin of each trace, in capillary electrophoresis, is not fixed, the apparent shift between the top and second-from-top traces, recorded in different runs, is 6 nucleotides, rather than the actual 4, though corresponding peak patterns in the two traces are readily apparent.

### DETAILED DESCRIPTION

The term “variable site of interest” is defined as a polymorphic site or SNP; or an insertion or deletion mutation.

The term “disambiguating” means resolving an ambiguity; which occurs if, based on the results in question, at least one sample in a particular pool cannot be identified as either normal or variant at a variable site of interest. Disambiguation encompasses any method of sequencing or genotyping or allele identification, including but not limited to using allele-specific primers.

The term “reaction steps” refers to steps involved in amplifying, labeling, or extending a primer, amplicon or oligonucleotide chain.

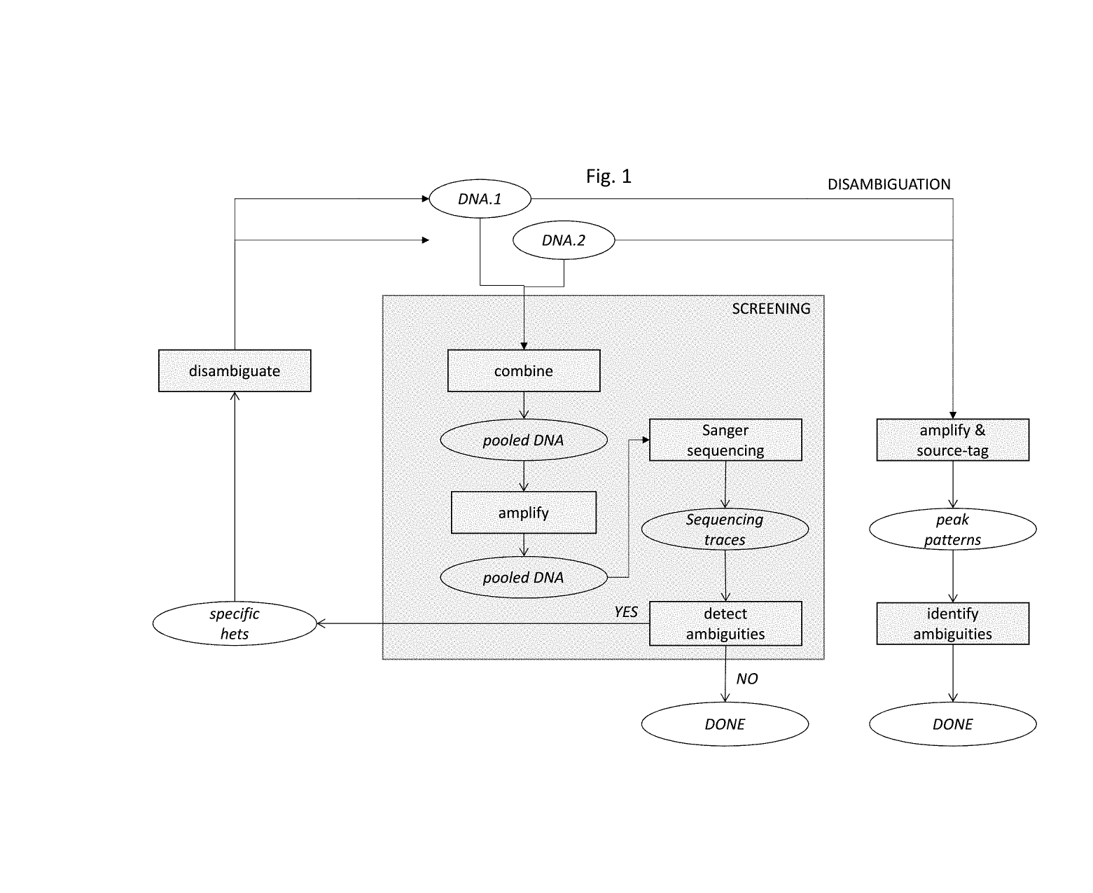

The present invention provides for the use of pooled Sanger sequencing as the first (“screening”) stage in allele profiling or sieving which may comprise a second (“disambiguation”) stage, as illustrated, for two samples, in FIG. 1. It also provides for the use of pooled Sanger sequencing, with specific source-tags and labeling primers, for disambiguation.

In a first embodiment, the present invention provides for combining two or more amplicons in a Sanger labeling (aka “cycle sequencing”) reaction using differentially fluorescently labeled dideoxy-nucleotides, and subsequent analysis of these pooled labeled products, preferably by capillary electrophoresis. As with standard Sanger sequencing, a single (contiguous) sequence (typically comprising a single exon) is analyzed. As with allele profiling and nucleic acid sieving, samples that contain at least one variant allele in one or more (known or unknown) position in the sequence, generally will introduce an ambiguity associated with two or more superimposed peaks at variable positions in the sequence.

However, contrary to the general practice of discarding as contaminated “double sequence” data (see “DNA Sequencing Troubleshooting Guide” by Eurofins Genomics, available on its website; “DNA SEQUENCING SANGER: TECHNICALS SOLUTIONS GUIDE” by Secugen, available on its website) the superimposed sequence traces generated by pooled samples may be decoded, so as to achieve disambiguation, as elaborated herein.

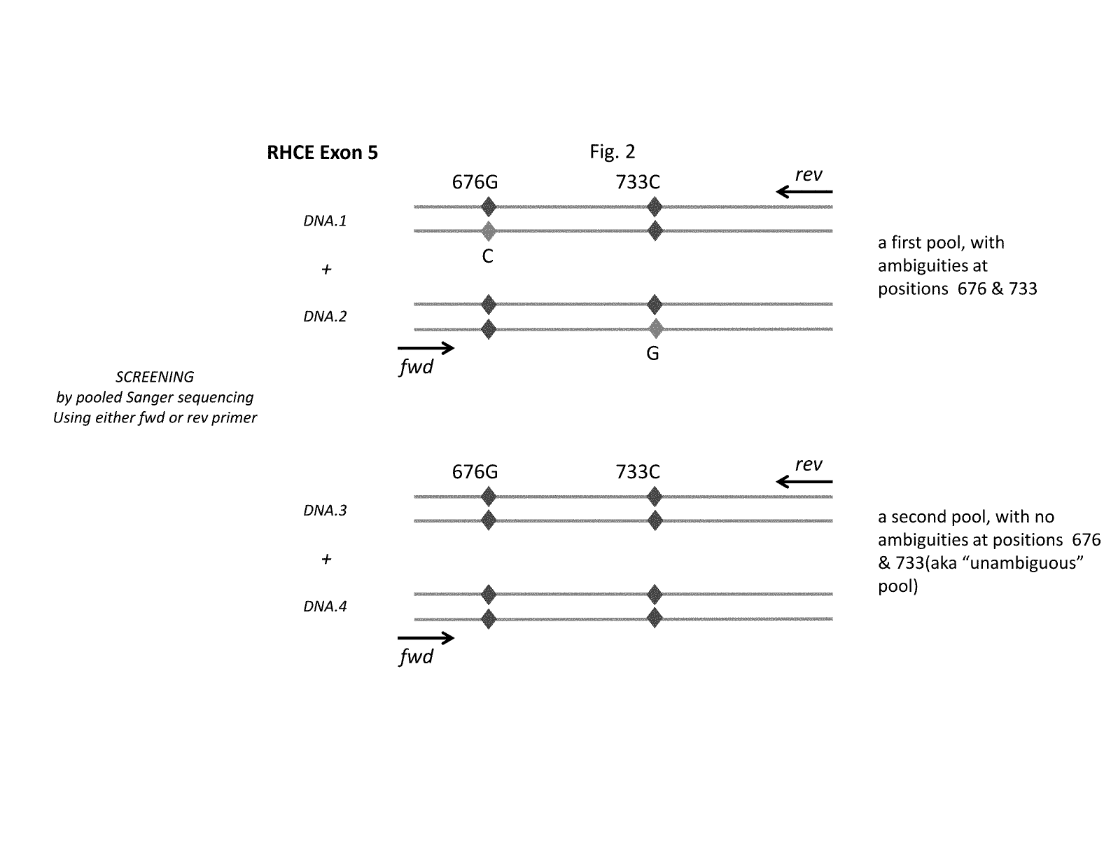

The process of the present invention will be a useful complement to the previously disclosed processes of allele profiling and nucleic acid sieving for the analysis of alleles and mutations, especially if all or the most prevalent mutations or alleles of interest reside on a single exon, as they do, in the case of mutations, for example, for β-thalassemia or for cystic fibrosis, or, in the case of alleles, for the polymorphic genes encoding the human leukocyte antigens (“HLA”) or the Rh antigens. More generally, the present invention also will be useful for analyzing a single amplicon (or other construct or reaction products) comprising multiple exons or sections thereof. Such amplicons may be generated by amplification with primer flanking the region in the sequence comprising a variable site, or multiple variable sites, of interest. Either of these flanking primers may be used in the subsequent Sanger labeling reaction of the combined samples, as illustrated in FIG. 2 for two polymorphisms in exon 5 of the RHCE gene, elaborated in Example 1. Special designs for labeling primers, for use in disambiguation, also are disclosed herein.

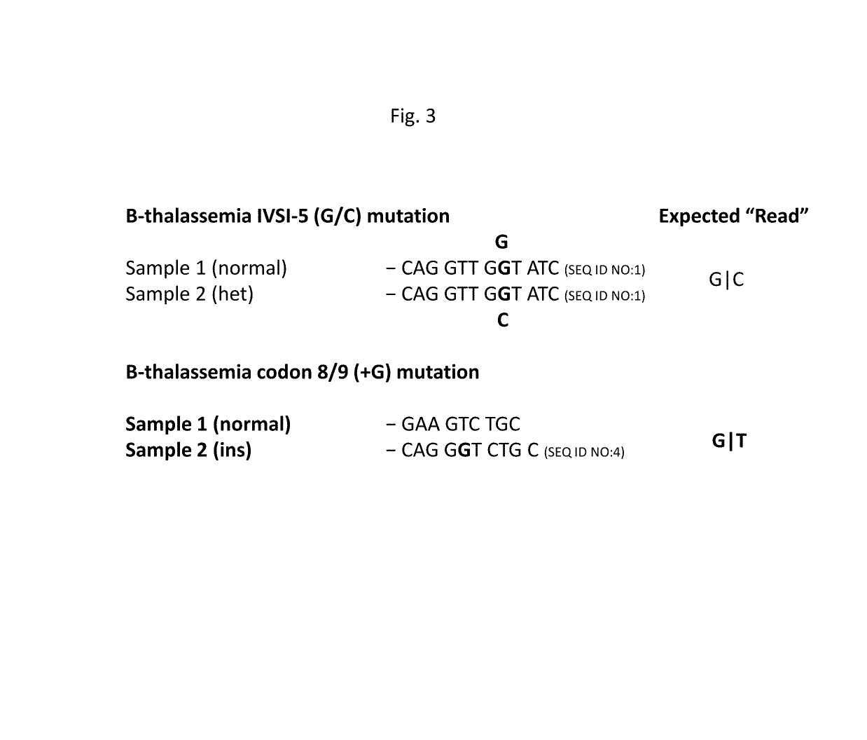

Thus, pooled Sanger sequencing of the β-thalassemia gene for a pool of two samples would produce, for IVSI-5 (G>C), the by far most commonly observed β-thalassemia mutation in Pakistanis (Ansari2011 “Molecular epidemiology of β-thalassemia in Pakistan: far reaching implications” Int J Mol Epidemiol Genet. 2(4): 403-408), the following expected “read” (see also, FIG. 3, upper panel):

```
                                                      G
Sample 1 (normal) CAG GTT GGT ATC (SEQ ID NO: 1)
Sample 2 (het) CAG GTT GGT ATC (SEQ ID NO: 1)
                                                      C
```

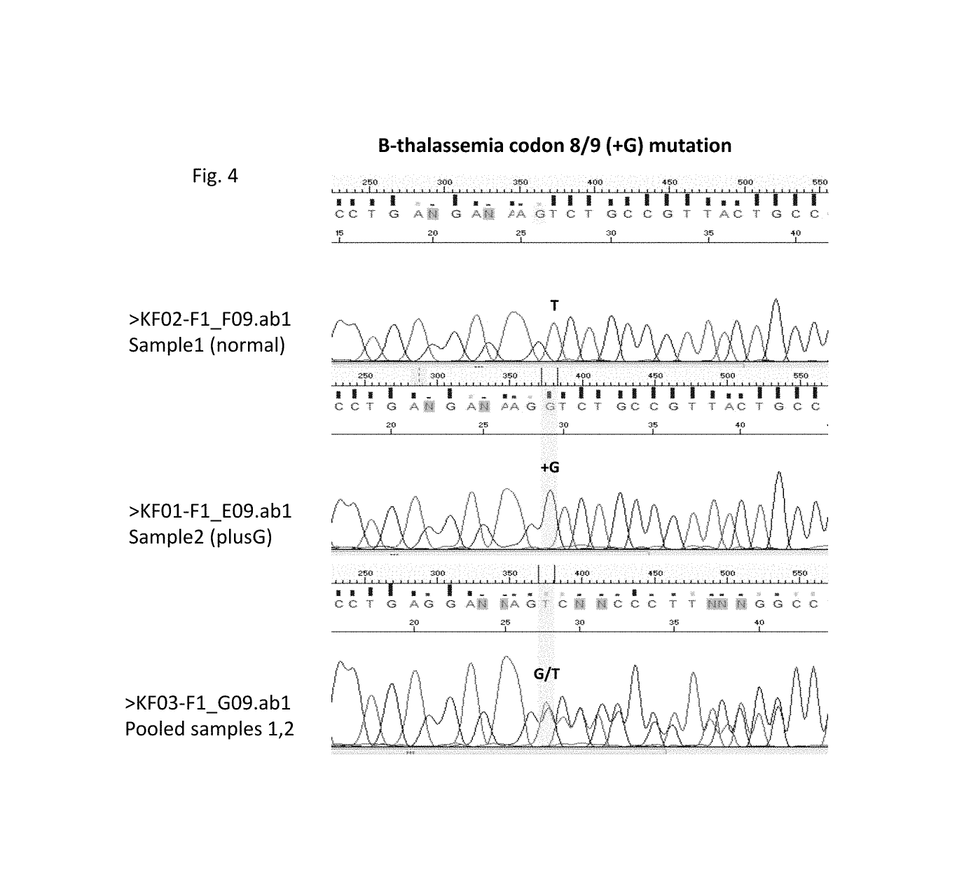

The presence of a mutation in the pool will be readily detected, in the form of a “het” signal, characterized here by peaks in two color channels; however, ambiguity generally will remain as to the identity of the sample(s) carrying the mutation, as the following separately pooled configurations: (GG|GC), (GC|GG), (GG|CC), and (CC|GG) all will produce the same heterozygous signature in the “compound” sequence trace (though peak intensities may provide additional information). Similarly, insertions and deletions are readily detected, as illustrated in FIG. 3 (lower panel) and elaborated with reference to FIG. 4 in Example 2. Pools displaying such ambiguities may be selected or de-selected, in accordance with desired allele patterns, as previously disclosed, or may be further analyzed to resolve ambiguities, as elaborated below.

Gain in Operational Efficiency—

The probability of encountering at least one heterozygous configuration in a pool of d samples may be estimated from the (assumed known) population frequencies of anticipated variant alleles (as discussed in the allele profiling and sieving references) so as to determine the optimal d, subject to the constraint that the d-fold dilution of samples incurred by pooling will set an upper limit, d≤dmax, to the extent of practical pooling.

In comparison to standard Sanger sequencing for mutation analysis, pooled Sanger sequencing will produce a gain reflecting the reduction in the number, N, of sequencing runs in the single sample format to a number not greater than N/d+d*(N/d)*prob (at least one mutation in d samples), where d denotes the number of samples in a pool. Assuming bi-allelic genes, prob (at least one mutation in 2*d alleles)=1−(1−f)^{2d} where f represents the probability that a sample comprises at least one of a set of variant alleles or mutations of interest.

For example, taking the carrier frequency for β-thalassemia to be f=1/30, reflecting the combined abundances of the most commonly observed mutations in South-Asian populations (Ansari2011), the expression yields 0.13, 0.23, 0.33 and 0.42, respectively, for d=2, 4, 6 and 8. Thus, for d=4, 96 β-thalassemia samples, combined into 96/4 pools, would yield 0.23*24 pools requiring disambiguation; if performed on individual samples (d=1), this scenario would entail performing an additional 0.23*24*4=22.8 or roughly 23 runs (assuming, in the worst case, that no two ambiguities are encountered in the same pool); thus, the total number of wells processed would be no greater than 96/4+23=47, a gain of roughly 2 (=96/47).

Allele profiling (including the use of source tags) would further reduce the number of requisite additional runs, by another factor of 4, to roughly 6 (=23/4), the factor of 4 reflecting the pooling of source-tagged first reaction products (as described in the allele profiling references).

A more detailed comparison would break out gains in first and second reactions—standard Sanger sequencing would require 96 first reactions (namely PCR amplification) plus 96 second reactions (namely: labeling), for a total of 192 reactions, the resulting fragments requiring 96 capillaries for analysis. In contrast, assuming d=4, allele profiling with pooled Sanger sequencing as the first stage, would require: 96/4 first reactions, 96/4 second reactions, analyzed in 24 capillaries, plus, for disambiguations: 23 first reactions plus 6 second reactions, analyzed in 6 capillaries.

Thus, the invention yields a substantial gain in process efficiency for carrier screening given the typically low carrier frequencies for inherited disorders. An example showing the application of pooled Sanger sequencing to molecular sieving for RHCE alleles is given below.

Disambiguation by Allele Profiling—

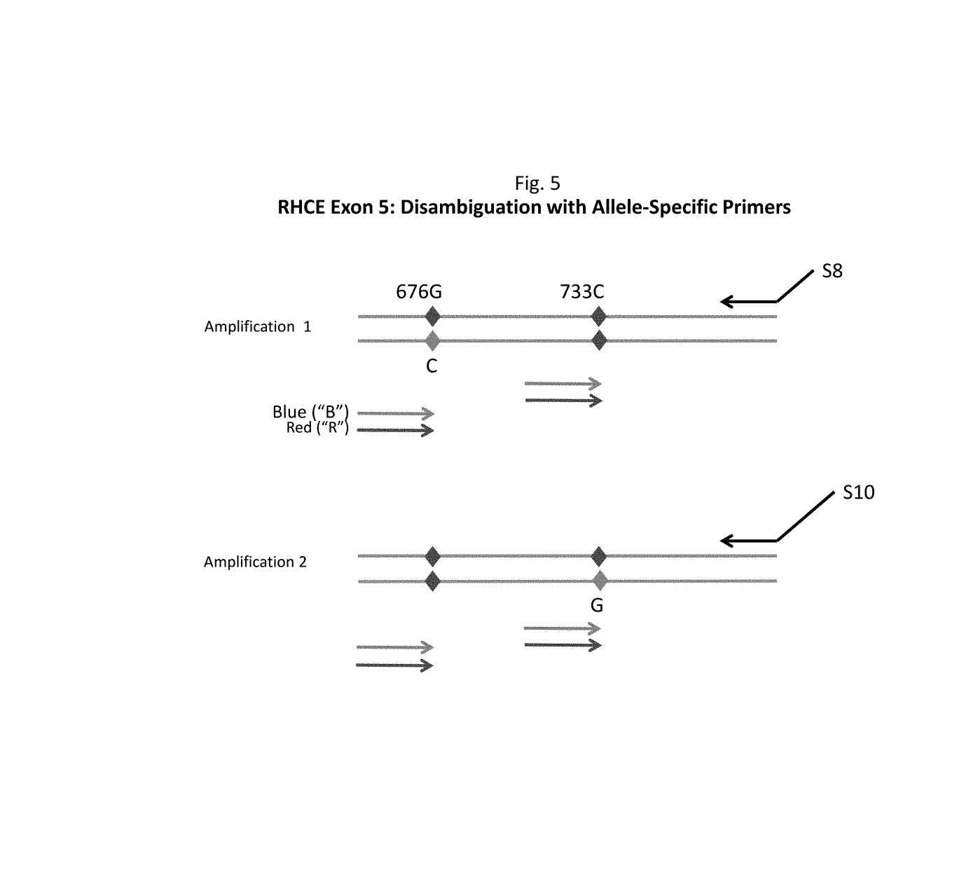

Ambiguities may be resolved, in accordance with the allele profiling process previously disclosed, by using allele-specific amplification at heterozygous positions of interest, either one at a time or several at a time. In a preferred embodiment, DNA from the constituent samples of ambiguous pools is amplified using one or more pairs of fluorescently labeled primers directed to the alleles at heterozygous positions, paired with source-tagged primers, as illustrated in FIG. 5, to produce source-and marker-tagged products which then are pooled for capillary electrophoresis. The configuration depicted in FIG. 5 would be expected to generate a total of 6 peaks, namely: 2 (one in the R, one in the B channel) for 676-S8; 1 for 733-S8; 2 for 676-S8; and 1 for 733-S8, thereby permitting the identification of alleles for each constituent sample and hence the resolution of ambiguities.

These allele-specific amplification reactions may be performed using genomic DNA from individual samples, or a set of amplicons independently generated by random priming of the genome or selected genomic regions of these samples. In the latter case, the amplification of a specific sequence of interest will be accomplished in a small number of cycles generating source-and-marker-tagged products.

Disambiguation by Pooled Sanger Sequencing of Source-Tagged Products—

In a further embodiment of the invention, the Sanger labeling reaction is performed with pools of first reaction products comprising source (“S”) tags, wherein source tags identifying the first reaction products produce predetermined relative shifts in the expected sequence traces, either by changing fragment length or (by one of several methods well known in the art) electrophoretic mobility.

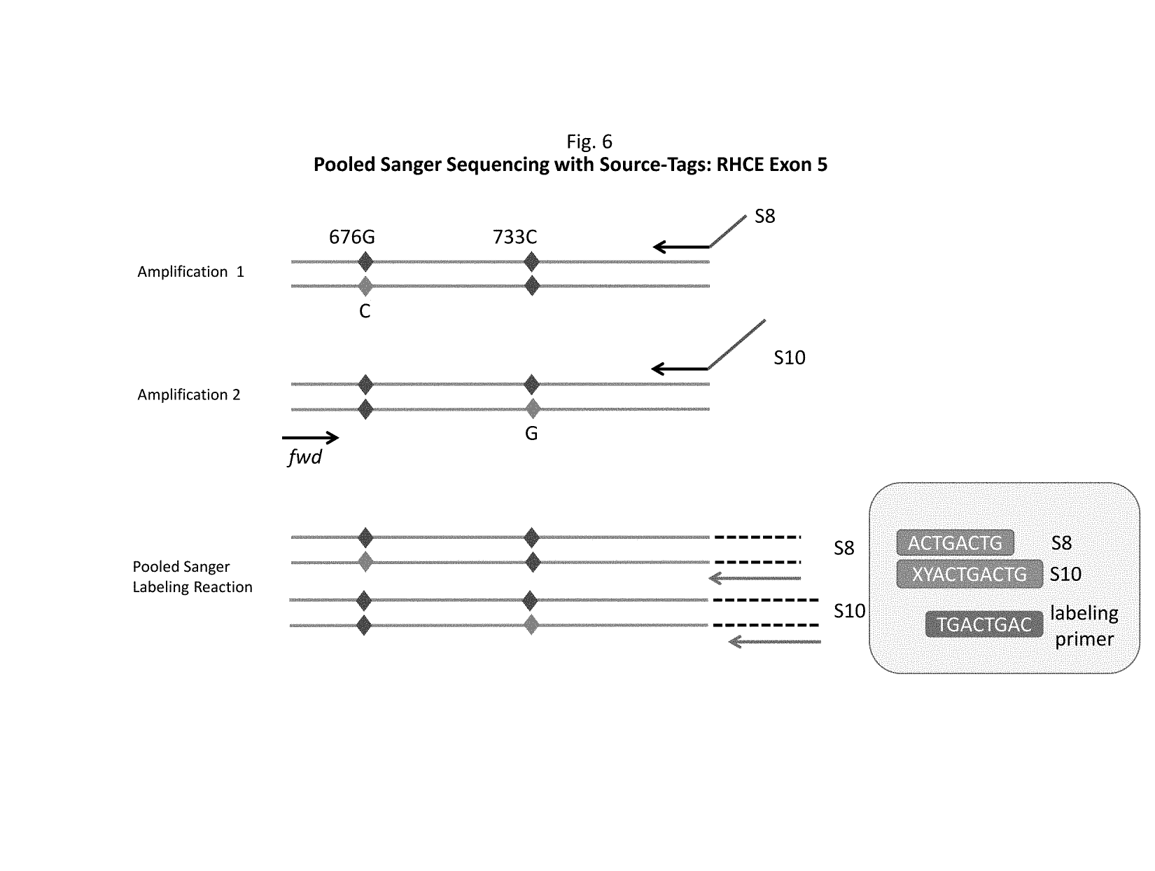

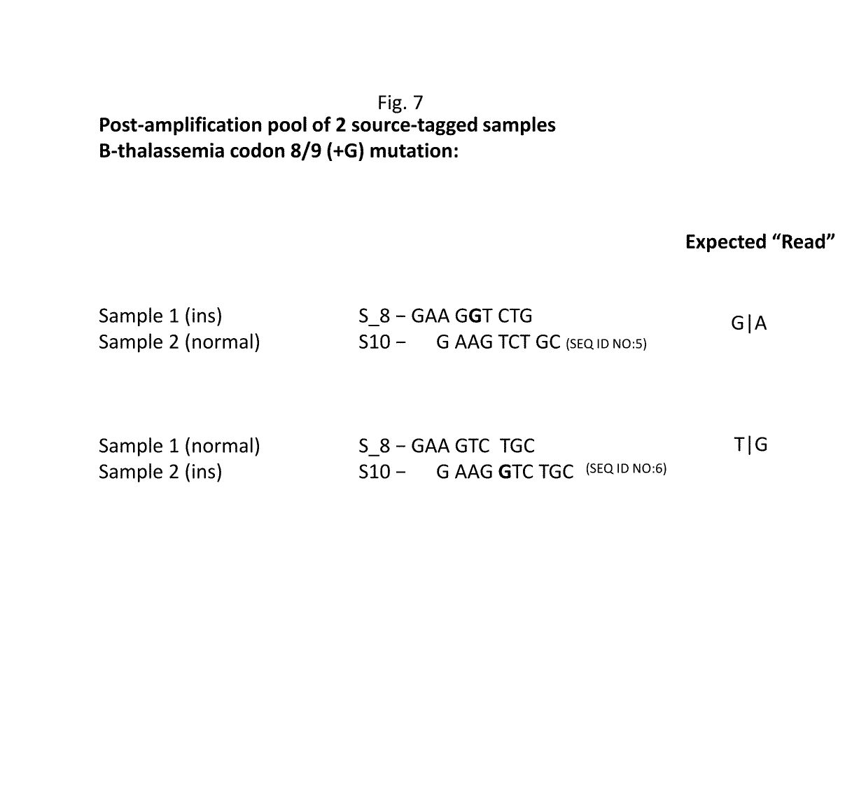

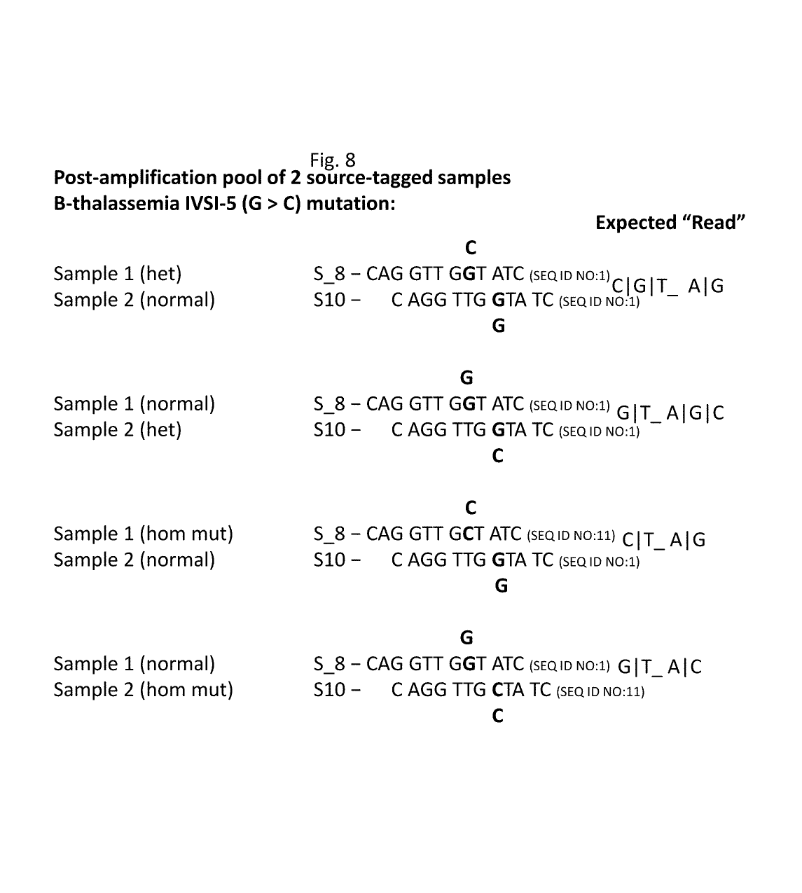

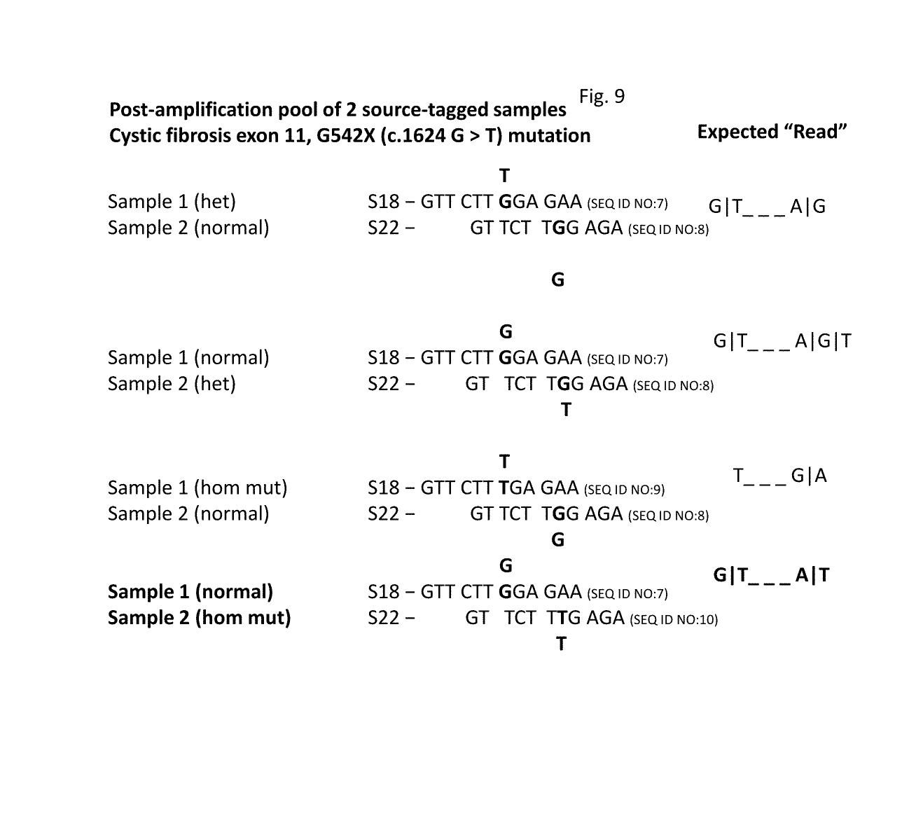

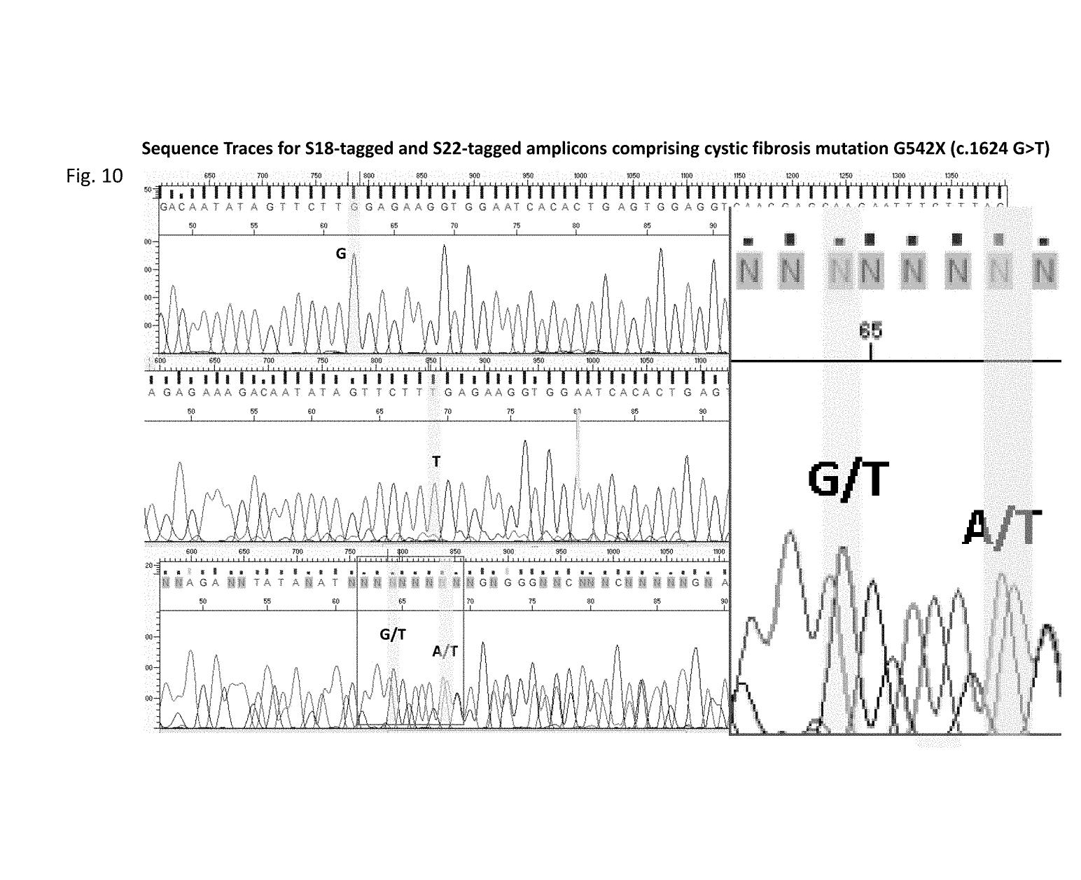

In one embodiment, to change fragment length, the source tags have a common 5′ subsequence which may comprise the entire sequence of the shortest tag, and the “universal” labeling primer is complementary to that subsequence, as illustrated in FIG. 6. This is elaborated, with reference to FIG. 7 and FIG. 8, in Example 3, for the β-thalassemia mutations IVS 1-5 (G>C) and codon 8/9 (+G) in (see above) and, with reference to FIG. 9 and FIG. 10, in Example 4. The resulting “reads,” while having the appearance of “contaminated” samples that would be rejected in standard Sanger sequencing practice may be decoded to resolve ambiguities. This is illustrated in the Examples below.

In another embodiment of the invention, the source tags differ in composition, by one or more base(s), at the end forming the junction with the gene- or exon-specific primer(s) as in FIG. 6, but, in contrast to the previous embodiment, unique corresponding labeling primers are designed with 3′ ends complementary to each unique primer-source tag junction. This embodiment permits two modalities of labeling, namely: first, using a combination of such labeling primers to label pooled source-tagged first reaction products; and second, using one or more (but not all) of the labeling primers to label one or more specific pooled first reaction product(s), thereby enabling disambiguation using pools of first reaction products, as only the corresponding individual sequence trace(s) will be generated. If the difference in composition between source tags extends over different numbers of bases, then peaks shift also may be introduced; this is elaborated with reference to FIG. 10 in Example 4.

Thus, given a post-PCR pool of first reaction products, the choice of labeling primer, in accordance with these embodiments, permits either screening of multiple such first products for ambiguities reflecting heterozygous configurations, or disambiguating such configurations, by introducing peak shifts and labeling only a subset of pooled first reaction products.

The process of the invention is in contrast to the analysis of Sanger sequence traces comprising signals from multiple samples by decomposition into constituents, with reference of a “dictionary” holding constituent peak patterns (or representations thereof). Superpositions of two or more such peak patterns are then compared to the observed pattern to infer the composition of the mixture, as shown for sequencing of the 16S rRNA gene for mixtures of bacterial pathogens (the Pathogenomix website; see also Kommeda12008 https://www.ncbi.nlm.nih.gov/pmc/articles/PMC2576573/pdf/0213-08.pdf). This approach is akin to that of decomposing genotypes into constituent alleles, where alleles are defined over multiple variable sites, step in the standard analysis of genotypes for highly polymorphic genes such as HLA.

Non-Integer Peak Shifts—

Ambiguity remains when the mutation or variable position in the shifted peak pattern of a second sample is superimposed on an identical base in the sequence of the first sample. For example, if, in FIG. 9, the sequence surrounding the IVSI-5 mutation were CAG GTC GGT ATC (SEQ ID NO:2), then a G-C configuration would appear in the compound trace, and this would be indistinguishable (but for possible peak intensity differences) from the peak pattern produced by a het configuration in Sample 1.

```
                                                                G
Sample 1 (normal) S_8-CAG GTC GGT ATC (SEQ ID No: 2)
Sample 2 (hom mut) S10-C AGG TCG CTA TC (SEQ ID No: 3)
                                                                  C
```

However, by constructing source-tags so as to introduce “non-integer” peak shifts (that is: shifts by non-integer multiples of the nominal peak-to-peak spacing of 1 base), this ambiguity is avoided: the presence of a second “C” peak, shifted by a non-integer displacement from the first, would unambiguously indicate the presence of a “het” configuration.

Non-integer peak shifts may be produced by using source tags that, for given length, differ in base composition. Thus, it has long been known that tag composition alters the electrophoretic mobilities of oligonucleotides (Frank 1979—“DNA chain length markers and the influence of base composition on electrophoretic mobility of oligodeoxyribonucleotides in polyacrylamide-gels” Nucleic Acids Research Vol. 8 pp. 2069-87). Alternatively, the fluorescent dyes used in commercial Sanger sequencing kits are well known to introduce differential peak shifts (requiring correction as a pre-processing step when aligning the traces recorded in the different color channels and normalization with respect to a “size ladder” included in each reaction). In addition, chemical modifications with “drag tags” also have been described. Other chemical modifications including methylation also are available to introduce peak shifts.

Key Process Steps—

The analysis of sequence traces recorded from a pool of source-tagged samples would proceed as follows:
detect all peaks (using a standard peak detection algorithm such as the multi-scale detection algorithm (Du et al., “Improved peak detection in mass spectrum by incorporating continuous wavelet transform-based pattern matching,” Bioinformatics, Vol. 22, pp. 2059-65 (2006).
correct peak positions for relative mobility shifts introduced by fluorescent dyes;
for all known or anticipated variable positions in the sequence(s):
   detect alleles for each such group of peaks; and
   resolve residual ambiguities by allele profiling or by using source-tagged primers and comparing “compound” sequence traces with expected “reads”.

### Examples

1—Detecting Variants by Pooled Sanger Sequencing: Sieving for RHCE Exon 5 Alleles

Exon 5 of this gene comprises several important alleles including (ISBT—https://tinyurl.com/vca97t93)

| Name | Polymorphism | Phenotype |
|---|---|---|
| A226P | 676G > C | e/E |
| V223F | 667 G > T | hrS-, others |
| Q233E | 697 C > G | Crawford |
| M238V | 712 A > G | “partial” c, e |
| L245V | 733 C > G | V, VS; partial c, e |

The administration of red cells that are not properly matched for the phenotype determined at this locus contributes to the risk of alloantibody formation. The rapid determination of especially the alleles at this locus, for recipients and donors or red cells, therefore has substantial clinical significance.

To apply the “screening” (or “sieving”) method of the invention using pooled Sanger sequencing, combine DNA samples from at least 2 individuals (d≥2) for amplification using standard primers flanking exon 5, where d is determined as disclosed; then, commit the resulting amplicons to the Sanger labeling reaction performed with either of the PCR primers, and analyze the resulting labeled products by capillary electrophoresis. As with molecular sieving generally, the abundance of the variant alleles in the table, for the population of interest, determines the probability of a variant and an associated ambiguity, and thus determines the expected number of pools that are unambiguous for one or more of the listed alleles. Constituent samples of these pools may be selected in accordance with desired allele patterns; for example, pools comprising candidate donor samples that are homozygous E—may be selected for immediate assignment to recipients with existing anti-E antibodies.

2—Detecting Variants by Pooled Sanger Sequencing: Screening for β-Thalassemia Mutations

Exon 1 of this gene comprises several of the most commonly observed mutations including substitutions, insertions and deletions. Illustrated here is the detection of an insertion in a pool of two samples of which one is normal, and the other is homozygous for the codon 8/9 (+G) mutation, producing the following expected “read” (see also FIG. 3, lower panel):

Sample 1 (normal)
GAA GTC TGC
Sample 2 (ins)
GAA GGT CTG

A “G-T” het configuration in the expected position of the insert, highlighted here by a bold-faced “G”, along with additional predictable downstream hets, indicates the presence of the insert in at least one allele. The corresponding sequence traces, in FIG. 4, indicate the presence of the insert, manifesting itself in the new “G/T” het at the highlighted position in the compound trace.

Analogously, deletions, such as the 4-base deletion in codon 41/42-CTTT, another common β-thalassemia mutation, would be readily detected by the appearance of predictable het configurations. A simple substitution will produce a characteristic het at the expected position.

The example illustrates the case d=2, with 2 copies of the variant allele in the pool. The value of d is limited only by detection sensitivity which must be such that 1 copy of a variant allele is reliably detected in a pool comprising 2d copies: thus, it is the detection sensitivity that ultimately limits the value of d≤dmax.

3—Disambiguation by Using Source Tags: β-Thalassemia Mutations—

As illustrated in FIG. 7, the ambiguity introduced in the pool by the codon 8/9 (+G) mutation is resolved by the introduction of source tags: the presence of the insert in Sample 1 will produce a G-A configuration at the expected position (along with additional predictable downstream “hets”), while the presence of the insert in Sample 2 will produce a G-T configuration at the expected position (along with additional predictable downstream “hets”). This approach is illustrated in FIG. 8 which lists expected “reads” produced by different instances of the IVS 1-5 (G>C) mutation. The presence of a “het” configuration in a first sample (“Sample 1”), with source-tag S8, but not in a second sample (“Sample 2”), with source tag S10, will produce 3 superimposed peaks (C-G-T) and 2 superimposed peaks (G-A), respectively, at the two expected positions in the “compound” trace recorded from the pool of the two samples, while the presence of a “het” configuration in “Sample 2,” but not in “Sample 1” will produce 2 superimposed peaks (G-T) and 3 superimposed peaks (A-G-C), respectively, at the expected positions in the “compound:” trace. These configurations are distinguishable from those produced by a sample that homozygous for the mutation, namely C-T and A-G (Sample 1 homozygous for mutation) versus G-T and A-T (Sample 2 homozygous for the mutation). For example, observation of the peak configuration “G-T” and “A-G-C” at the expected variable positions in the two constituent samples, will identify the configuration of the constituent samples as homozygous normal (sample 1) and “het” (sample 2).

4—Disambiguation by Using Source Tags: Cystic Fibrosis Mutations—

A further example is that of assigning the G542X (G>T) mutation in exon 11 of the cystic fibrosis gene. Of the expected sequence “reads” for four possible configurations, shown in FIG. 9, the corresponding “compound” sequence trace, in FIG. 10 (bottom panel and insert) reveals the configuration “G-T_A-T”, thereby unambiguously identifying Sample 1, tagged with S18, to be normal and Sample 2, tagged with S22, to be homozygous for the mutation. As with the earlier example, discussed with reference to FIG. 6, the Sanger labeling reaction creating the “compound” sequence was performed with a universal labeling primer directed against a shared subsequence of tags S18 and S22.

It is worth pointing out that the use of a labeling primer directed to the S22 tag sequence permits the labeling of only the S22-tagged sample, even when both samples are in the pool: in fact, this is how the trace in the middle panel of FIG. 10 was generated. Source-tagged primers that share a 5′ subsequence, but comprise unique 3′ subsequences, at least 1 nucleotide in length, will permit universal labeling, to generate the “compound” trace, or differential labeling, to generate specific sequences from pooled samples.

1-3. (canceled)

4. The process of claim 24 wherein the source tags are designed, and labeled second reaction products are formed with labeling primers selected, such that peaks representing the labeled di-deoxynucleotide-terminated fragments are shifted by a known amount.

5. The process of claim 24 wherein peak position are determined by capillary electrophoresis.

6. The process of claim 24 wherein the selected sample pools are unambiguous for the desired allele because all constituent samples are homozygous for the desired allele.

7. The process of claim 24 wherein the selected sample pools are ambiguous for the desired allele because at least one constituent sample is not homozygous for the said desired allele.

8. (canceled)

9. The process of claim 24 wherein the desired alleles include markers for genes from the following list: β-thalassemia, cystic fibrosis, HLA, and RH.

10. The process of claim 24 wherein determining the presence of a desired allele at the variable site(s) of interest is by determining the presence of one of at least two peaks, at specific positions corresponding to said variable site(s) of interest.

11. The process of claim 10 wherein the determining if any desired alleles are in any combined pool is by determining whether at the position of variable site(s) of interest, a single peak (in a single color channel) or at least two peaks (in at least two color channels) are observed in the combined pools.

12-23. (canceled)

24. A process of selecting subsets of nucleic acid samples having one or more desired alleles of interest at one or more variable sites of interest, or not having any desired alleles, wherein the presence of said desired alleles gives rise to certain labeled reaction products, the process comprising:
(a) for each of one or more desired alleles:
(i) determining a value, d, representing a first maximum number of samples to be combined into pools, by finding first that the probability of any pool having any of the desired alleles does not exceed a predetermined probability threshold, wherein said desired alleles are known to occur in the population at a specified frequency, and wherein if some of the one or more desired alleles occur at specified frequencies substantially different than the specified frequencies of other desired alleles, forming different sample pools for determining if any of said desired alleles having said substantially different frequencies are in any pool, wherein d is determined in accordance with said substantially different frequencies;
(ii) if d for said desired alleles is greater than a preset upper limit dₘₐₓ, then setting the value d for said desired alleles equal to dₘₐₓ;
(iii) if d for said desired alleles is less than 1, then setting the value of d for said desired alleles equal to 1.
(b) performing the following steps:
(i) combining aliquots from the nucleic acid samples to form a plurality of sample pools with not more than d samples per pool;
(ii) associating particular said desired alleles in different sample pools with a source tag identifying the different sample pools;
(iii) amplifying genomic regions of the samples containing the desired alleles to generate amplicons including source-tags;
(iv) combining aliquots from one or more of the different amplicon-containing pools to form one or more combined pools wherein the number of amplified samples in each combined pool does not exceed dₘₐₓ;
(v) forming labeled reaction products from said amplicons using labeled di-deoxynucleotides which thereby generates labeled reaction products, but wherein only a subset of the labeled reaction products allow identification of desired alleles;
(vi) determining if any pool contains desired alleles by identifying the label(s) of said subset of labeled reaction products; and
(c) identifying the source tags of samples having, or not having, desired alleles, to determine the sample pool(s) of origin for any such samples, and selecting particular sample pool(s) containing at least one sample having a desired allele; or, selecting particular sample pool(s) having no sample including a desired allele.

25. The process of claim 24 wherein, in the event of ambiguity, steps b(v) and b(vi) are repeated with a single primer directed to a specific subsequence of the source-tag incorporated in the amplicons such that only selected amplicons, but not the other amplicons, form labeled reaction products.

26. The process of claim 24 wherein source tags are designed to share common 5′ subsequences.

27. The process of claim 24 wherein the labeling of combined amplicons is with a single primer directed to a common 5′ subsequence of the source tags.

28. The process of claim 24 wherein the labeling of a selected amplicon, but not the other amplicons within the pool of combined amplicons, is with a single primer directed to a specific subsequence of the source-tag incorporated in that amplicon.

29. The process of claim 28 wherein said single primer includes a unique 3′ subsequence, of at least 1 nucleotide in length.


SEQUENCE LISTING (11 sequences)

SEQ ID NO: 1 - DNA, 12 bp, Artificial Sequence
  Description of Artificial Sequence Synthetic oligonucleotide
caggttggtatc

SEQ ID NO: 2 - DNA, 12 bp, Artificial Sequence
  Description of Artificial Sequence Synthetic oligonucleotide
caggtcggtatc

SEQ ID NO: 3 - DNA, 12 bp, Artificial Sequence
  Description of Artificial Sequence Synthetic oligonucleotide
caggtcgctatc

SEQ ID NO: 4 - DNA, 10 bp, Artificial Sequence
  Description of Artificial Sequence Synthetic oligonucleotide
cagggtctgc

SEQ ID NO: 5 - DNA, 9 bp, Artificial Sequence
  Description of Artificial Sequence Synthetic oligonucleotide
gaagtctgc

SEQ ID NO: 6 - DNA, 10 bp, Artificial Sequence
  Description of Artificial Sequence Synthetic oligonucleotide
gaaggtctgc

SEQ ID NO: 7 - DNA, 12 bp, Artificial Sequence
  Description of Artificial Sequence Synthetic oligonucleotide
gttcttggagaa

SEQ ID NO: 8 - DNA, 11 bp, Artificial Sequence
  Description of Artificial Sequence Synthetic oligonucleotide
gttcttggaga

SEQ ID NO: 9 - DNA, 12 bp, Artificial Sequence
  Description of Artificial Sequence Synthetic oligonucleotide
gttctttgagaa

SEQ ID NO: 10 - DNA, 11 bp, Artificial Sequence
  Description of Artificial Sequence Synthetic oligonucleotide
gttctttgaga

SEQ ID NO: 11 - DNA, 12 bp, Artificial Sequence
  Description of Artificial Sequence Synthetic oligonucleotide
caggttgctatc